(19) United States
(12) Patent Application Publication
(10) Pub. No.: US 2024/0190303 A1
(43) Pub. Date: Jun. 13, 2024

(54) Safety Seat for Emergency Vehicles

(71) Applicant: DEMERS, AMBULANCE MANUFACTURER INC./ DEMERS, MANUFACTURIER D’AMBULANCES INC., Beloeil (CA)

(72) Inventors: Gilbert Dubocquet, Saint-Jean-Sur-Richelieu, Qc (CA); Hélène Trahan, Beloeil, Qc (CA); Martin Laliberté, Beloeil, Qc (CA); Alain-Olivier Boudreau, St-Jean-sur-Richelieu, Qc (CA); Pierre Rondeau, Montréal, Qc (CA)

(21) Appl. No.: 18/554,831

(22) PCT Filed: Apr. 11, 2022
(86) PCT No.: PCT/CA2022/000016

§ 371 Date: Oct. 11, 2023

Related U.S. Application Data
(60) Provisional application No. 63/173,564, filed on Apr. 12, 2021.

(51) Int. Cl. B60N 2/07; B60N 2/40; B60N 2/68

(52) U.S. Cl. CPC B60N 2/072; B60N 2/40; B60N 2/688

(57) ABSTRACT

Described herein is a safety seat for emergency vehicles including a structure including a generally L-shaped column defining a backrest structure and a seat structure; a transversal element provided at a junction of the backrest structure and the seat structure.

The present disclosure relates to safety seats. More specifically, the present disclosure is concerned with a safety seat for emergency vehicles.

### BACKGROUND

Safety seats for emergency vehicles and, more particularly, safety seats adapted for installation in an ambulance or other emergency vehicle to enable medical attendants to safety perform emergency medical services on a patient while the vehicle is in motion are known in the art.

These safety seats are generally designed to withstand important acceleration forces since they must protect the medical attendant seated therein should a road accident occur. Accordingly, the structure of these seats is often bulky, and yield a big bulky seat that is heavy.

Another problem with conventional safety seats for emergency vehicles in the size and shape of the seat portion of the safety seat that is generally not suited for medical attendants that often have to lean forward to perform medical services.

### BRIEF DESCRIPTION OF THE DRAWINGS

in the appended drawings:

FIG. 1 is a perspective view of an assembled safety seat for emergency vehicle according to an illustrative embodiment;

FIG. 2 is a perspective view similar to FIG. 1 but showing the safety seat structure in dashed lines;

FIG. 3 is a side elevational view of the safety seat of FIG. 1;

FIG. 4 is a perspective view of the safety seat structure according to a first illustrative embodiment;

FIG. 5 is a sectional view taken along line 5-5 of FIG. 4;

FIG. 6 is an exploded view of the safety seat of FIG. 1:

FIG. 7 is a perspective view of the column and transversal elements of a safety seat structure according to a second illustrative embodiment.

FIG. 8 a sectional view taken along line 8-8 of FIG. 7;

FIG. 9 is a partially exploded view of the safety seat structure of FIG. 7;

FIG. 10 is a perspective view of a safety seat structure according to a third illustrative embodiment;

FIG. 11 is a perspective view of the column of a safety seat structure according to a fourth illustrative embodiment; and

FIG. 12 is a perspective view of the column of a safety seat structure according to a fifth illustrative embodiment;

FIG. 13 is a front perspective view of a safety seat structure according to a sixth illustrative embodiment;

FIG. 14 is a sectional view taken along line 14-14 of FIG. 13;

FIG. 15 is a rear perspective view of the safety seat structure of FIG. 13:

FIG. 16 is a perspective view illustrating the seat to base connecting mechanism in a locked configuration; and

FIG. 17 is a perspective view illustrating the seat to base connecting mechanism in an unlocked configuration.

### DETAILED DESCRIPTION

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”). “having” (and any form of having, such as “have” and “has”), “including” (and any form of including, such as “include” and “includes”) or ‘containing’ (and any form of containing, such as “contain” and ‘contains’), are inclusive or open-ended and do not exclude additional, unrecited elements or process steps.

In the present specification and in the appended claims, various terminology which is directional, geometrical and/or spatial in nature such as “longitudinal”, “horizontal”, “front”, rear”, “upwardly”, “downwardly”, etc. is used. It is to be understood that such terminology is used for ease of description and in a relative sense only and is not to be taken in any way as a limitation upon the scope of the present disclosure.

The expression “connected” should be construed herein and in the appended claims broadly so as to include any cooperative or passive association between mechanical parts or components. For example, such parts may be assembled together by direct coupling, or indirectly coupled using further parts.

Other objects, advantages and features of the safety seat for emergency vehicles will become apparent upon reading of the following non-restrictive description of illustrative embodiments thereof, given by way of example only with reference to the accompanying drawings.

Generally stated, an illustrative embodiment is concerned with a safety seat for emergency vehicles including a structure including a generally L-shaped column defining a backrest structure and a seat structure. A transversal element is provided at a junction of the backrest structure and the seat structure and a strengthening bracket is connected to both the backrest structure and the seat structure. The strengthening bracket may be integral or separated from both the backrest structure and the seat structure. The safety seat includes a seat mounted to the seat structure and a backrest mounted to the backrest structure.

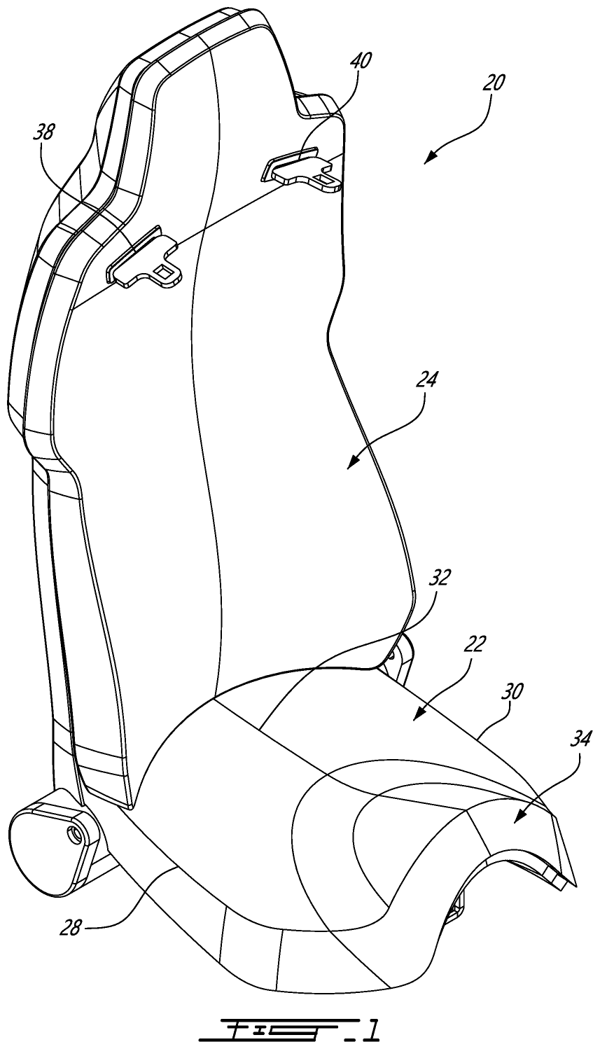

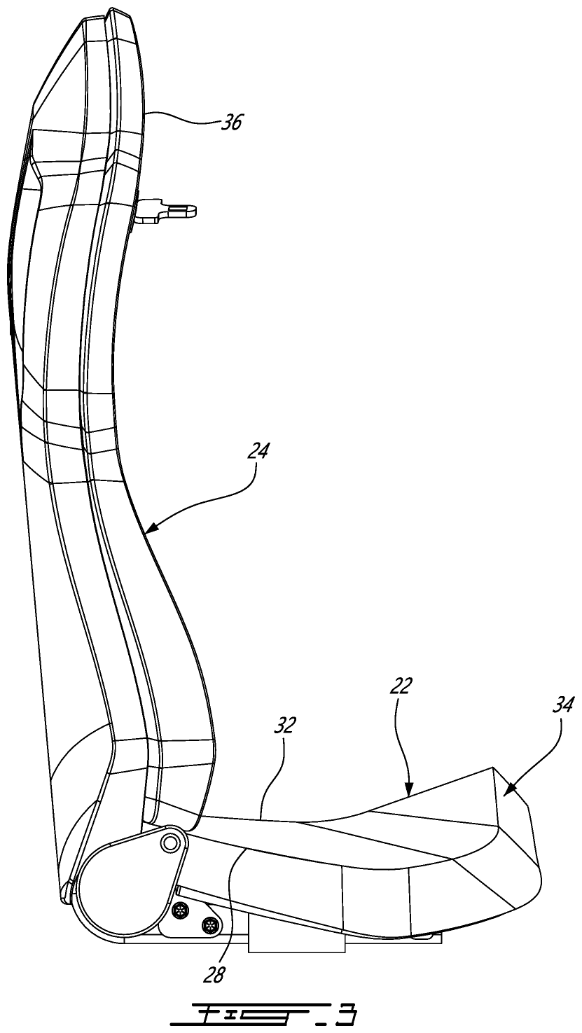

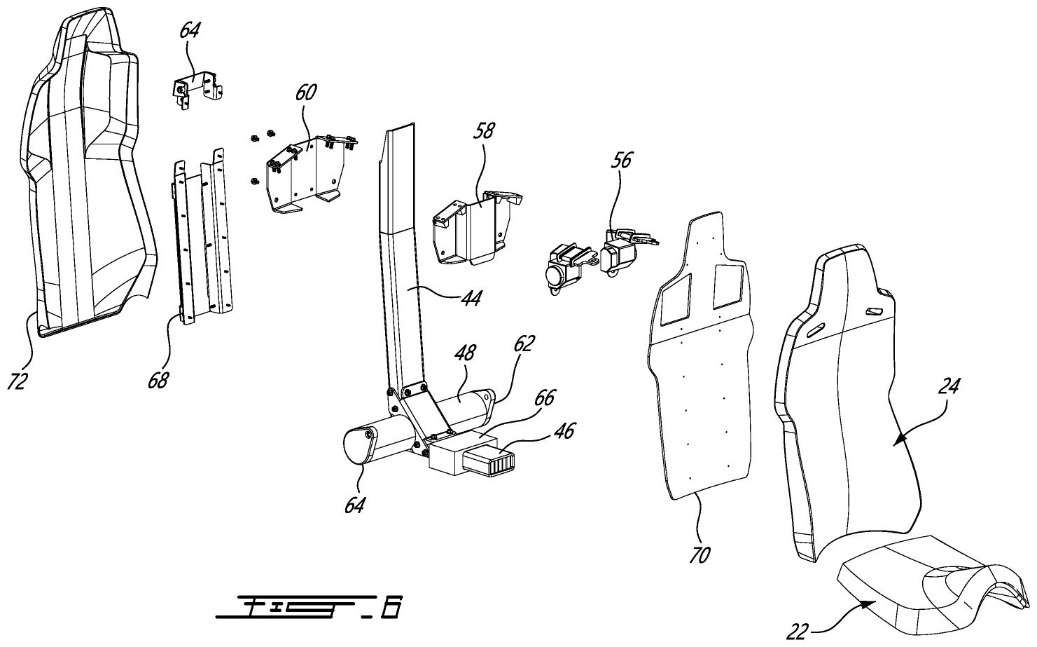

Turning now to FIGS. 1 to 6 of the appended figures, a safety seat 20 for emergency vehicles according to a first illustrative embodiment will be described.

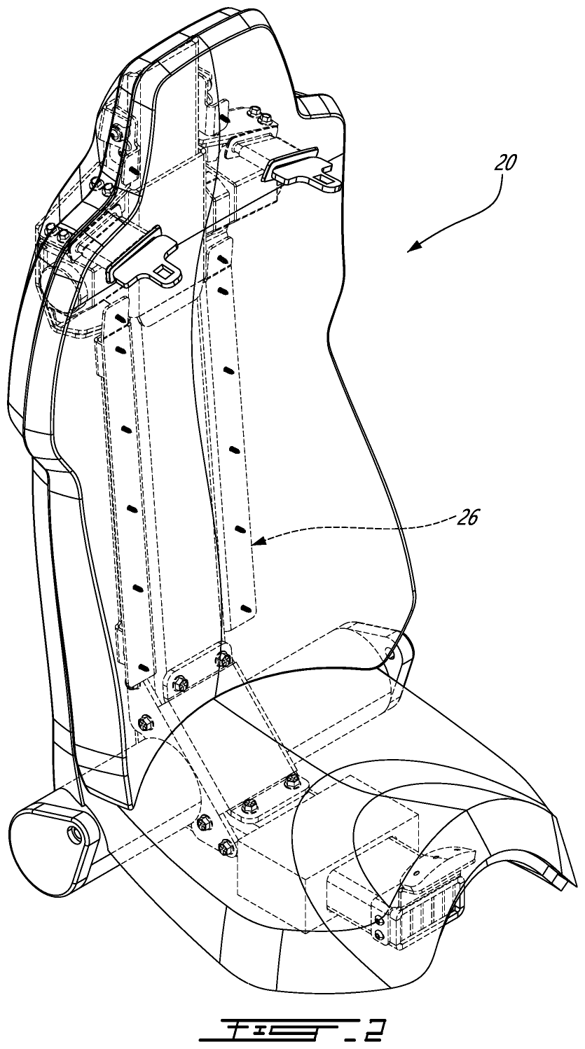

As can be better seen from FIGS. 1 to 3, the safety seat 20 includes a saddle-type seat 22 and a backrest 24. To secure these elements to one another and eventually to a floor base (not shown), a safety seat structure 26 is shown in dashed lines in FIG. 2.

The saddle-type seat 22 has a generally inverted U-shaped front profile, therefore sloping the lateral sides 28 and 30 lower than the middle 32 of the seat. Furthermore, a horn 34 centrally provided in the front of the seat place the legs of the occupant in a stable slightly spread configuration. Paired with the sloping lateral sides 28 and 30, the horn 34 places the occupant in a stable position where the occupant can easily bend forward to perform emergency medical services on a patient while maintaining a stable position.

The backrest 24 includes a headrest portion 36 provided with safety belt passages 38, 40 on either side thereof.

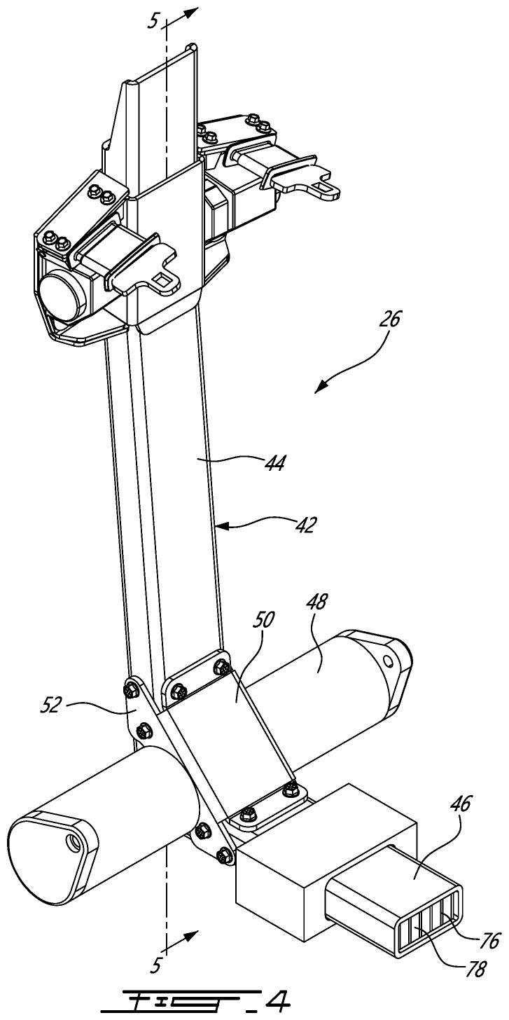

Turning now to FIGS. 4 to 6 of the appended drawings, the safety seat structure 26 according to a first illustrative embodiment will be described.

The safety seat structure 26 includes a generally L-shaped column 42 defined by a backrest structure 44 integrated with a seat structure 46. A transversal element 48, here in a cylindrical form, is provided at the junction of the backrest and seat structures 44 and 46. Strengthening brackets 50 and 52, separate from the structures 44 and 46, reinforce the connection between these structures as will be described hereinbelow.

The safety seat structure 26 also includes a safety belt assembly 54 mounted to the column 42 in the vicinity of the headrest 36. Conventionally, the safety belt assembly 54 includes two belt rollers 56 mounted to either sides of the column 42 via a front bracket 58 and a rear bracket 60. Of course, fasteners are used to mount the brackets 58 and 60 to one another.

The safety belt assembly also includes an abdominal part (not shown) that is attached to the end caps 62, 64 of the transversal elements 48 that are tear-drop shaped and include an aperture to fasten the bottom part of the safety belt assembly.

As will be understood by one skilled in the art, the end caps 62, 64 could be replaced by supplemental belt rollers (not shown) to yield a four point harness.

A seat mounting bracket 66 is mounted to the seat structure 46 and is so configured to receive the seat 22 thereon. The bracket 66 is also used to mount the safety seat 20 to a seat base (not shown).

A backrest mounting bracket assembly configured to allow the backrest 24 to be mounted to the backrest structure 44 includes rear brackets 68, 69 and a front backrest support 70. Once these elements are fastened to the backrest structure 44, the backrest 24 can be secured thereto. It is to be noted that a back cover 72 is mounted to the backrest 24 to hide the structure and to provide a smooth back surface.

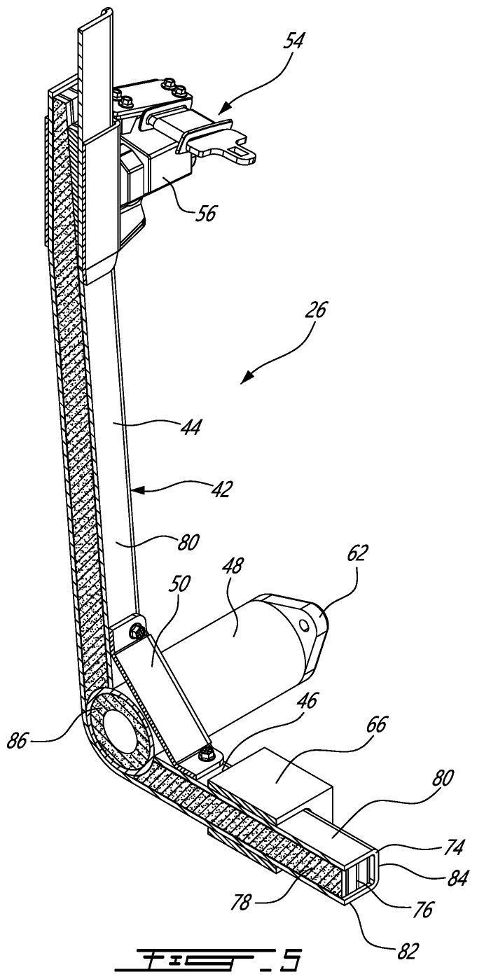

Turning now more specifically to FIG. 5 of the appended drawings, the L-shaped column 42 is defined by a generally rectangular tubing 74. It has been found that 6061-T6 aluminum is adequate for this tubing. Of course, other materials could be used.

The tubing 74 is filled with alternated layers of carbon fiber sheets 76 and of corelite foam 78, from the company Corelite Composites. These layers 76 and 78 are snugly inserted in the tubing 74 to thereby increase the stiffness thereof by filling any gap therein.

One skilled in the art will understand that the carbon fiber layer 76 could be replaced by other structuring materials such as, for example aluminum 7075-T6.

Similarly, the structural foam layer 78 could be made from other gap filling materials.

The generally rectangular tubing 74 includes a front wall 80, a rear wall 82 and lateral walls 84. As can be seen from FIG. 5, portions of the front wall 80 and of the lateral walls 84 are removed at the junction of the backrest structure 44 to the seat structure 46 to allow passage of the transversal element 48. The rear wall 82 is curved to fit around the element 48. The transversal element 48 is made of the same material as the rectangular tubing 74 and includes a tube of corelite foam 86 therein to increase its stiffness.

One skilled in the art will understand that the front wall 80 can be welded to the transversal element 48 at junctions thereof.

A shallow circumferential channel (not shown) can be done on the outer surface of the transversal element 48 to accept the lateral walls 84 and therefore correctly position the transversal element 48 with respect to the column 42 during assembly thereof.

A front strengthening bracket 50 and two lateral strengthening brackets 52 (only one shown in FIG. 4) are provided to maintain the orientation of the backrest structure 44 with respect to the seat structure 46. These brackets are made of the same material as the rest of the structure and are mounted via regular fasteners. Of course, these brackets 50 and 52 could also be welded or otherwise securely mounted to the structure.

One skilled in the art will also understand that the strengthening brackets 50 and 52 could be integral, i.e., made from one folded piece.

Having a column-type structure to support the seat and backrest is interesting for many reasons: it allows energy absorption from different directions; it can absorb vibrations generated by the movement of the vehicle; and it is lighter than conventional safety seat structures.

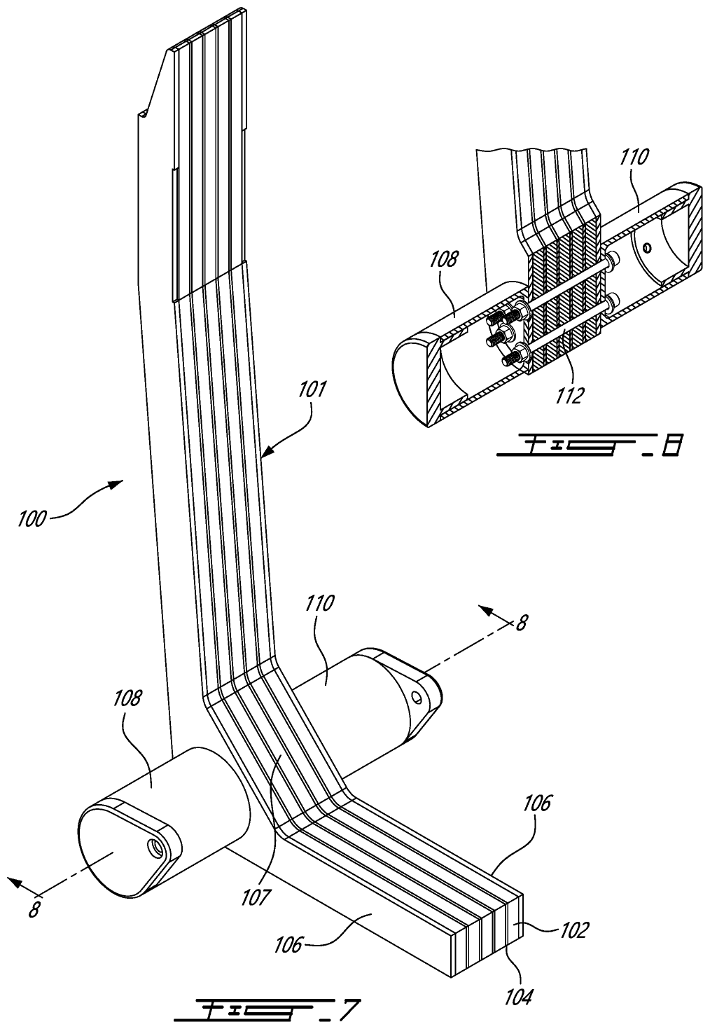

Turning now to FIGS. 7 to 9 of the appended drawings, a safety seat structure 100 according to a second illustrative embodiment will be described. It is to be noted that for concision purpose, only the differences between the structure 100 and the structure 26 of FIGS. 1 to 6 will be discussed hereinbelow.

Generally stated, the structure 100 has a generally L-shaped column 101 that lacks the generally rectangular tube configuration of the structure 26 described hereinabove. The alternate layers of corelite 102 and of carbon fiber 104 forming the column 101 are bonded to one another and lateral layers of aluminum 106 are provided. It has been found that aluminum 7075T6 has been found suitable to form the layers 106. Of course, other materials could be used.

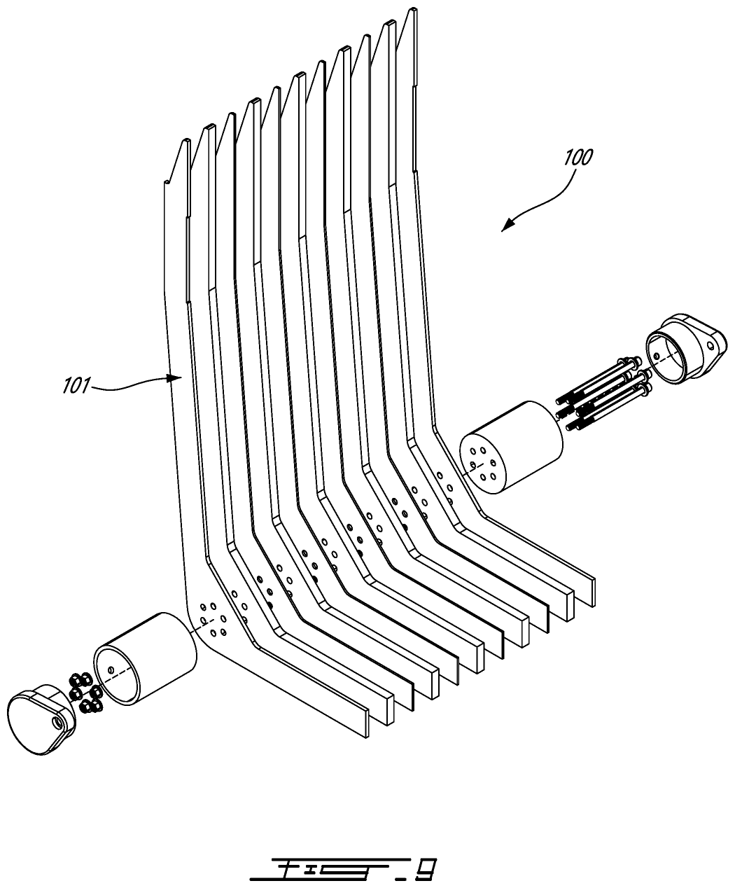

As can be seen from FIG. 8, the transversal element 48 of FIGS. 1 to 6 has been replaced by two transversal elements 108, 110 that are mounted to the column 101 using conventional fasteners 112 going through the column 101.

Returning to FIG. 7, one skilled in the art will understand that the junction of the backrest structure 103 to the seat structure 105 includes an integral bracket 107 to stiffen the column 101 by providing more material at the Junction thereof.

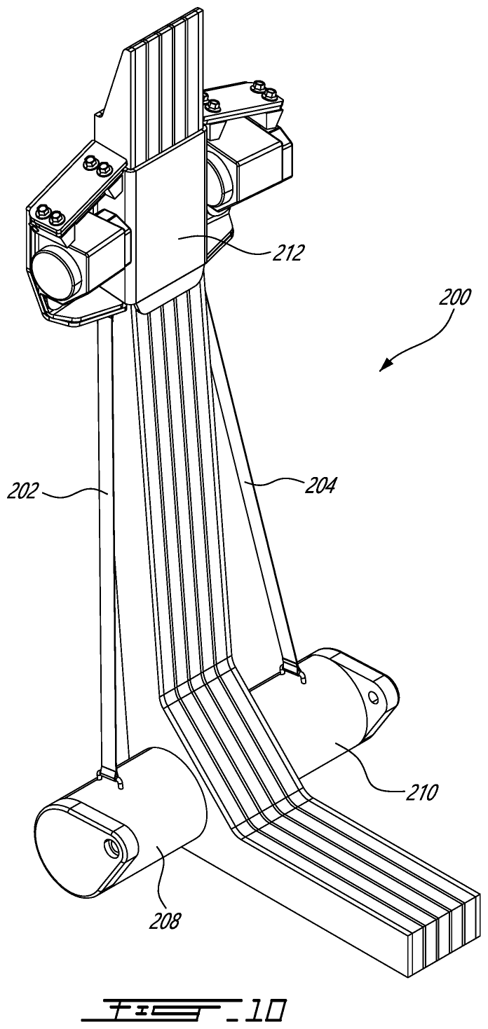

FIG. 9 shows the safety seat structure 100 in an exploded view.

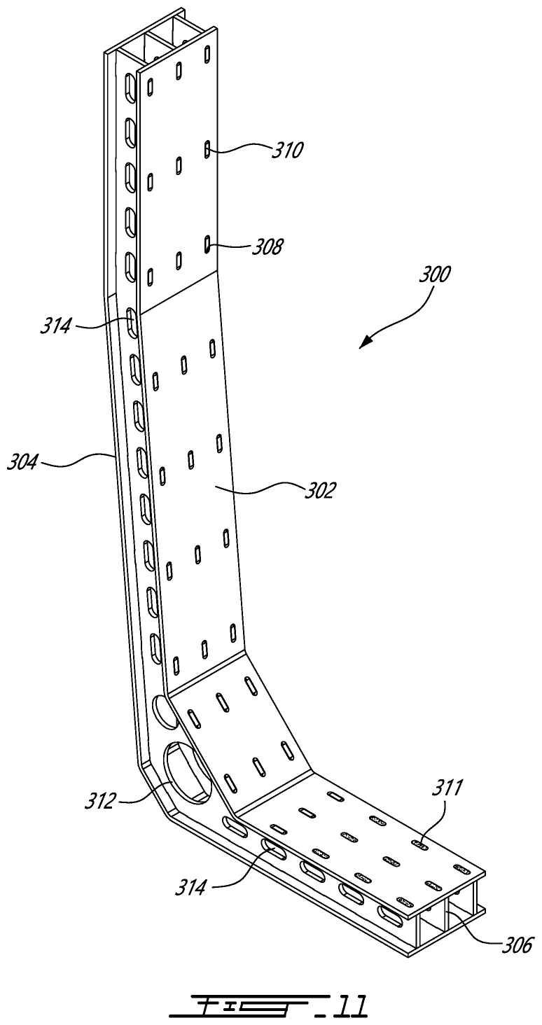

Turning now to FIG. 10 of the appended drawings, a safety seat structure 200 according to a third illustrative embodiment will be described. Since the structure 200 is very similar to the structure 100 illustrated in FIGS. 7 to 9, only the differences thereof will be described hereinbelow, for concision purpose.

Generally stated the main difference is that the structure 200 includes guy straps 202 and 204 respectively provided between the lateral elements 208, 210 and the safety belt assembly 212. These straps 202, 204 help strengthening the backrest structure in case of lateral impacts.

Of course, the number, position and nature of the straps can be modified.

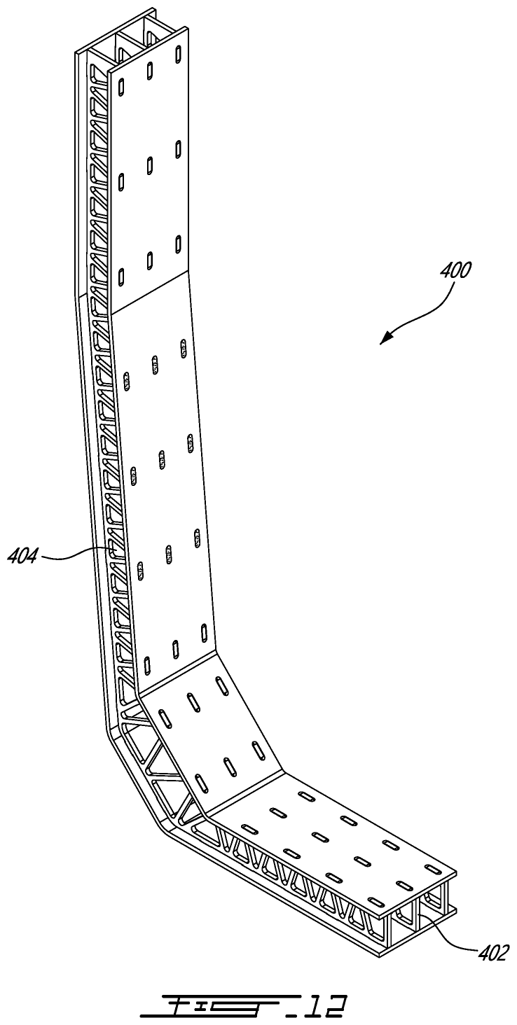

Turning now to FIG. 11 of the appended drawings a generally L-shaped column 300 according to a fourth illustrative embodiment will be described.

The column 300 is entirely made of aluminum plates. A folded front plate 302 and a folded rear plate 304 are maintained in a spaced apart relationship by three L-shaped plates 308. The front and back plates 302 and 304 are provided with oblong apertures 308 while the L-shaped plates 306 include corresponding tabs 310 so positioned and configured as to enter the apertures 308 to allow welding of these parts together (see welds 311).

Each of the L-shaped plates 306 include a circular aperture 312 allowing the transversal element (not shown) therethrough and other oblong apertures 314 provided to reduce the overall weight of the column 300.

It is to be noted that while three L-shaped plates 306 are illustrated herein, this number could be changed.

It has been found that 7075T8 type aluminum is an adequate material to make the L-shaped column 300.

As will be understood by one skilled in the angled portion of the front plate 302 may be viewed as an integral strengthening bracket since it strengthens the interconnection of the backrest structure to the seat structure. The conforming shape of the L-shaped plates 306 also serve as strengtheners between the backrest structure and the seat structure.

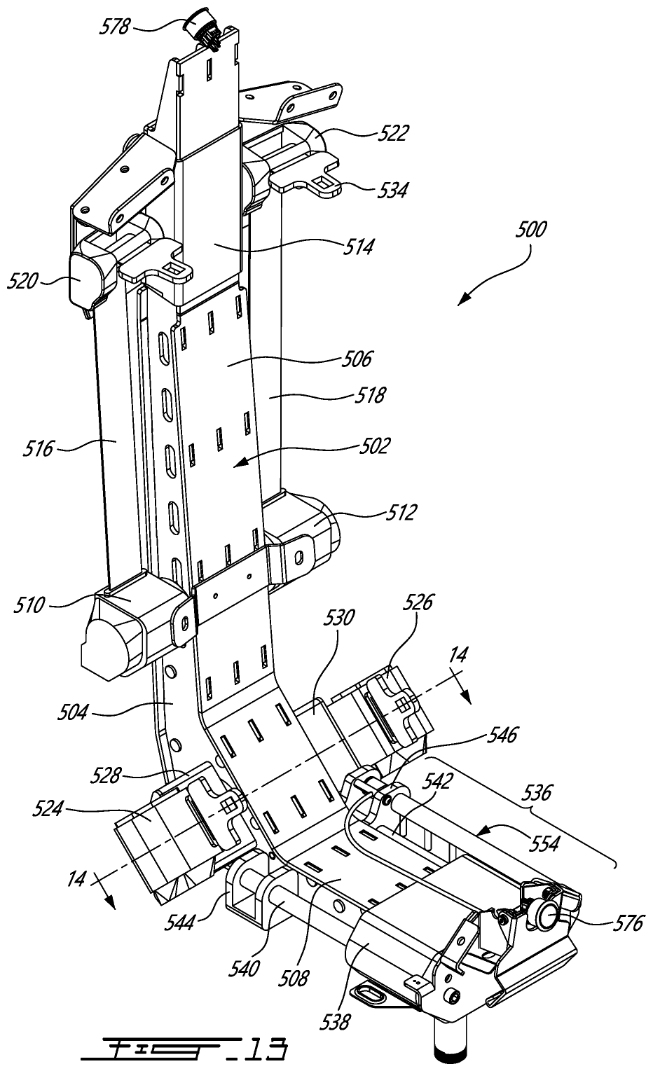

Turning to FIG. 12 of the appended drawings a generally L-shaped column 400 according to a fifth illustrative embodiment will be described.

The main difference between the column 400 and the column 300 of FIG. 11 is the shape and number of apertures of the L-shaped plates 402. Indeed, the apertures 404 are generally triangular and more numerous. The column 400 could be viewed as a triple I-beam.

Furthermore, the column 400 does not include a circular aperture allowing the transversal element (not shown) therethrough. When such a column is used separate transversal elements such as the elements 108 and 110 of FIG. 8 are used.

Again, as will be understood by one skilled in the art, the angled portion of the front plate of the column 400 and the shape of the plates 402 may be viewed as an integral strengthening brackets since they strengthen the interconnection of the back structure to the seat structure.

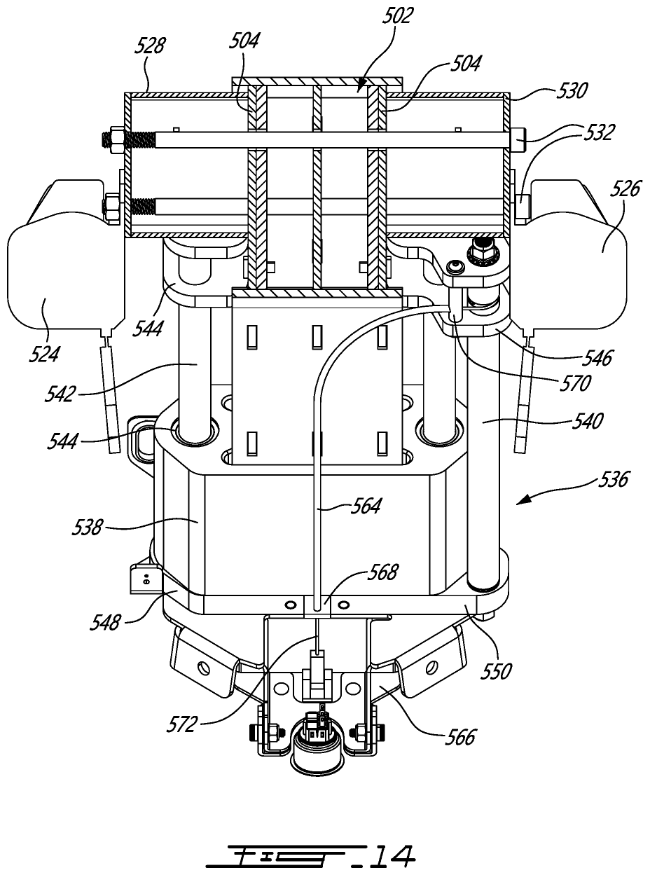

Turning now to FIGS. 13 to 18, a safety seat structure 500 according to a sixth illustrative embodiment will be described. Since this structure 500 is similar to the structures described hereinabove, and for concision purpose, only the differences will be described hereinbelow.

Generally stated, the structure 500 has a column 502 similar to the column 300 of FIG. 11 but includes supplemental separate lateral strengthening brackets 504 to strengthen the junction between the backrest structure 506 and the seat structure 508. These brackets 504 are mounted to the column 300 via conventional fasteners but could also be welded thereto.

The backrest structure includes two belt rollers 510, 512 that are provided lower than the headrest portion 514. The belts 516 and 518 pass through respective guiding elements 520, 522 to allow the free ends thereof to be in the vicinity of the headrest portion 514 when not in use.

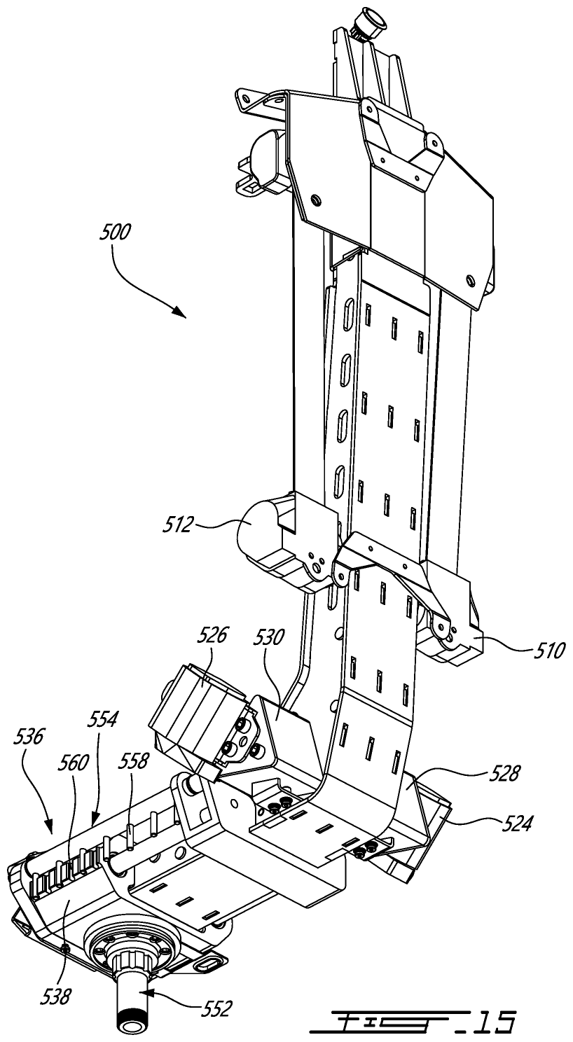

The safety seat structure 500 also includes two supplemental belt rollers 524, 526 respectively mounted to transversal elements 528, 530. As can be better seen from FIG. 14, the transversal elements 528 and 530 are secured to the column via fasteners 532 (only two shown) going through the brackets 504 and the column 502. Some of the fasteners 532 also secure the rollers 524 and 526 thereto.

One skilled in the art will understand that while the four belts shown herein are provided with seat belt clips such as 534, one of the belts could advantageously be provided with a seat belt buckle assembly (not shown) configured to receive the clips from the other three belts.

To increase the available movements of the emergency worker using the seat mounted to the safety seat structure 500, a selectively actuated sliding mechanism 536 is used to mount the structure 500 to a seat base (not shown) usually fixedly mounted to the floor of the emergency vehicle (also not shown).

More specifically, the sliding mechanism 536 is mounted to the seat structure 508 so as to allow longitudinal movements of the seat. The sliding mechanism 536 includes a body 538 mounted to the seat structure 508 via two longitudinal rods 540, 542, as can be better seen from FIG. 14. Of course, bearings, such as 544, are provided between the body 538 and the rods 540, 542. The rods 540, 542 are mounted to the seat structure 508 via brackets 544-550.

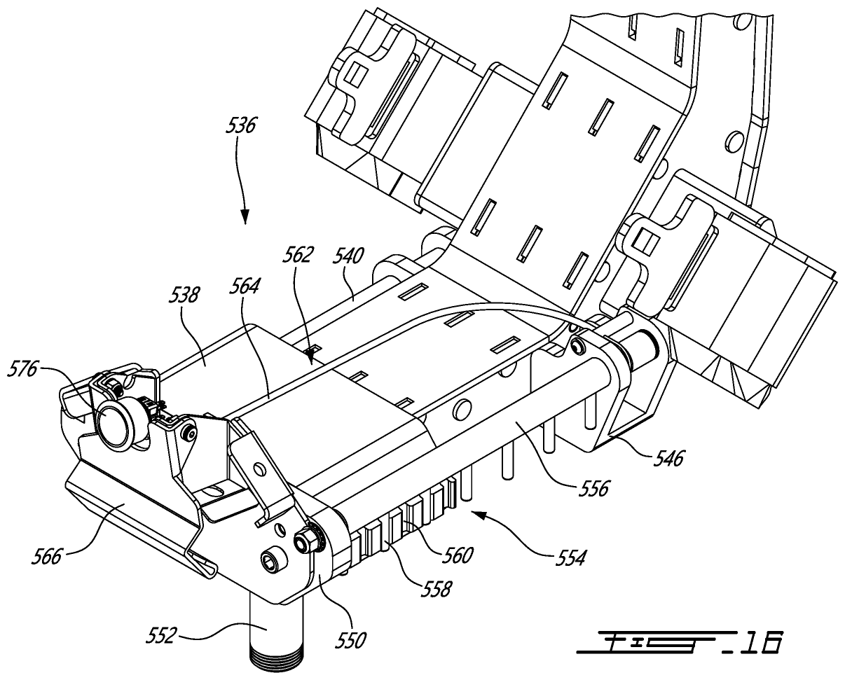

As can be better seen from FIG. 15, a post 552 is mounted to the underside of the body 538 to mount the seat structure 500 to the seat base (not shown).

The sliding mechanism 536 also includes a locking arrangement 554 to prevent sliding movements by default and to selectively allow movements when the user allows it.

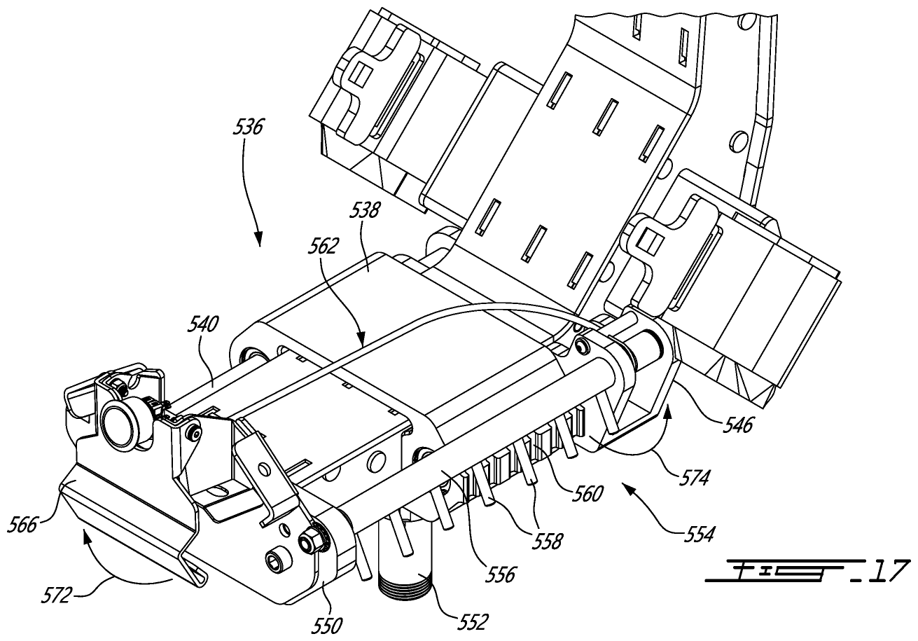

As can be better seen from FIGS. 16 and 17, the locking arrangement 554 includes a pivotable rod 556 mounted to and between the brackets 546 and 550. The rod 556 includes equally spaced projections 558 while the body 538 includes corresponding projection receiving channels 560.

FIG. 16 illustrates the locking arrangement 554 in its locked position, i.e., when the projections 558 are positioned in the channels 560. When it this position, the body 538 is mechanically prevented from longitudinally slide on the rods 540, 542.

A user actuated mechanical linkage assembly 562 is provided to pivot the rod 556 so as to momentarily release the projections 558 from the channels 560. The mechanical linkage assembly 562 includes a Bowden cable 564 mounted between a user accessible handle 566 and the pivoting rod 556. As can be better seen from FIG. 14, the housing of the Bowden cable 564 is mounted between a first bracket 568 provided in the vicinity of the handle 556 and a second bracket 570 mounted to the bracket 546.

One end of the inner cable 571 is mounted to the handle 566 while the other end thereof is partially rolled about the pivoting rod 556.

Accordingly, one skilled in the art that by pulling on the handle 566 (se arrow 572 in FIG. 17) the rod 556 is pivoted (see arrow 574) to thereby release the projections 558 from the channels 560 and thereby place the locking arrangement in an unlocked position and allow the body 538 to slide on the rods 540, 542.

While not shown herein, the pivoting rod 558 includes a biasing mechanism that forces the rod 556 towards its locked position when the handle 566 is released by the user. This biasing mechanism could, for example, include springs provided between the rod 556 and the brackets 546, 550.

One skilled in the art will notice that the spacing between the channels 560 is half the spacing between the projections 558 to allow more locking positions.

The safety seat structure 500 also includes first and second momentary switches 576 and 578 that can be used to control the seat-base mechanism (not shown).

One skilled in the art will understand that features of the various above-described embodiments could be provided on other embodiments. As non-limiting examples, the guy-straps of the column 200 and/or the sliding mechanism 536 could be provided on any of the other embodiments described herein.

As will easily be understood by one skilled in the art, the type of seats that can be mounted to the safety seat structures described hereinabove can be different than the saddle-type seat 22 shown in FIGS. 1 to 8.

It is to be understood that the safety seat for emergency vehicles is not limited in its application to the details of construction and parts illustrated in the accompanying drawings and described hereinabove. The safety seat for emergency vehicles is capable of other embodiments and of being practiced in various ways. It is also to be understood that the phraseology or terminology used herein is for the purpose of description and not limitation. Hence, although the safety seat for emergency vehicles has been described hereinabove by way of illustrative embodiments thereof, it can be modified, without departing from the spirit, scope and nature thereof

What is claimed is:

1. A safety seat structure for emergency vehicles including a generally L-shaped column defining a backrest structure and a seat structure; a transversal element provided at a junction of the backrest structure and the seat structure; and a strengthening bracket associated to both the backrest structure and the seat structure.

2. A safety seat structure as recited in claim 1, wherein the backrest structure, the seat structure and the bracket generally define a triangle through which the transversal element passes.

3. A safety seat structure as recited in claim 1, wherein the L-shaped column is defined by a generally rectangular tubing.

4. A safety seat structure as recited in claim 1, wherein the L-shaped column includes bonded L-shaped layers of carbon fibers alternating with L-shaped layers of rigid gap filing material; L-shaped lateral layers of aluminum are bounded thereto, the L-shape of the layers is so configured as to define an integral strengthening bracket.

5. A safety seat structure as recited in claim 1, wherein the L-shaped column includes a folded front plate, a folded rear plate and L-shaped plates provided between the front and rear plates; the folded front and rear plates and the L-shaped plates are so configured as to define an integral strengthening bracket.

6. A safety seat structure as recited in claim 5, wherein the folded front and back plates are provided with apertures aligned with corresponding tabs of the L-shaped plates to register the L-shaped plates with the front and back plates; the tabs and the apertures are welded.

7. A safety seat structure as recited in claim 5, further comprising generally L-shaped lateral strengthening plates mounted to the L-shape plates.

8. A safety seat structure as recited in claim 5, wherein the folded front plate, the folded rear plate and the L-shaped plates are made of 7075T6 type aluminum.

9. A safety seat structure as recited in claim 5, wherein the L-shaped plates include weight reducing apertures.

10. A safety seat structure as recited in claim 1, wherein the transversal element traverses the L-shaped column.

11. A safety seat structure as recited in claim 1, wherein the transversal element includes first and second elements laterally mounted to the L-shaped column.

12. A safety seat structure as recited in claim 1, further comprising a sliding mechanism associated with the seat structure; the sliding mechanism is so configured as to move longitudinally along the seat structure;
the sliding mechanism including a post configured to me mounted to a seat base.

13. A safety seat for emergency vehicles including:
a structure including a generally L-shaped column defining a backrest structure and a seat structure; a transversal element provided at a junction of the backrest structure and the seat structure; a strengthening bracket connected to both the backrest structure and the seat structure;
a seat mounted to the seat structure, and
a backrest mounted to the backrest structure.

14. A safety seat as recited in claim 13, further comprising a sliding mechanism associated with the seat structure, the sliding mechanism is so configured as to move longitudinally along the seat structure; the sliding mechanism including a post configured to me mounted to a seat base.

15. A safety seat as recited in claim 13, wherein the seat is a saddle-type seat.
